(19) United States
(12) Patent Application Publication
(10) Pub. No.: US 2025/0003623 A1
(43) Pub. Date: Jan. 2, 2025

(54) AIR PURIFIER

(71) Applicant: Dyson Technology Limited, Wiltshire, Malmesbury (GB)

(72) Inventors: Stephen Benjamin COURTNEY, Gloucester (GB); Benjamin David CARLESS, Gloucester (GB)

(21) Appl. No.: 18/709,058

(22) PCT Filed: Oct. 17, 2022
(86) PCT No.: PCT/GB2022/052635

§ 371 Date: May 10, 2024

(30) Foreign Application Priority Data
Nov. 10, 2021 (GB) 2116172.4

(51) Int. Cl. F24F 11/79; B01D 46/00; B01D 46/46; F24F 8/108; F24F 11/77; F24F 120/12

(52) U.S. Cl. CPC F24F 11/79; B01D 46/0047; B01D 46/46; F24F 8/108; F24F 11/77; F24F 2120/12

(57) ABSTRACT

An air purifier is described that includes a blower to generate an airflow, a filter to remove pollutants from the airflow, and a nozzle having an outlet through which the filtered airflow is emitted. The air purifier further includes an actuator assembly to adjust the direction of the filtered airflow, an image sensor to capture image data, and a control unit to detect a face in the image data and to control the actuator assembly such that the filtered airflow is directed at the face.

### FIELD OF THE INVENTION

The present invention relates to an air purifier.

### BACKGROUND OF THE INVENTION

Air quality is a growing concern for many people, with a variety of airborne pollutants having known or suspected harmful effects on human health. Air purifiers can be used to filter the air within a room. Whilst this may lead to a reduction in the overall level of pollutants within the room, the level of pollutants may continue to be high at specific locations within the room, perhaps where occupants are situated. Furthermore, with the opening and closing of doors and windows, maintaining effective purification within a room can be difficult.

### SUMMARY OF THE INVENTION

The present invention provides an air purifier comprising: a blower to generate an airflow; a filter to remove pollutants from the airflow; a nozzle comprising an outlet through which the filtered airflow is emitted; an actuator assembly to adjust the direction of the filtered airflow; an image sensor to capture image data; and a control unit to detect a face in the image data and to control the actuator assembly such that the filtered airflow is directed at the face, wherein the filtered airflow has an exit velocity at the outlet of no greater than 1 m/s.

Rather than trying to achieve bulk filtration of air within a room, the air purifier of the present invention instead directs a jet of filtered air at the face of user. As a result, the air breathed by the user may have a lower level of pollutants. Conceivably, a conventional air purifier may be directed at the face of a user. However, there are at least two difficulties with this. First, continual adjustment of the direction of the air purifier would be required to ensure that, as the user moves, the filtered air continues to be directed at the face of the user. However, continually adjusting the air purifier, at least manually, would not be a sustainable or realistic solution. Second, the airflow emitted from a conventional air purifier has a relatively high exit velocity. The relatively high exit velocity is required to ensure that the filtered air is circulated well within the room. If the exit velocity were relatively low, the filtered air would be localised around the air purifier, and thus the air purifier would provide relatively poor efficacy. However, as a consequence of the high exit velocity, directing the airflow of a conventional air purifier at the face of a user is likely to be uncomfortable, particularly over a prolonged period of time. For example, the user may find that the continuous, high movement of air over the face is unpleasant. In particular, the high movement of air may cause drying of the eyes and/or mouth.

The air purifier of the present invention automatically tracks the face of the user. In particular, the control unit detects the face in the image data captured by the image sensor, and controls the actuator assembly such that the filtered airflow is directed at the detected face. Consequently, as the face of the user moves, the air purifier continues to direct a jet of filtered airflow at the face without the need for manual intervention. In addition to tracking the face of the user, the filtered airflow emitted from the nozzle has an exit velocity no greater than 1 m/s. As a result, the velocity of the filtered airflow at the face of the user is also relatively low. This then has the benefit that the air purifier is likely to be more comfortable, particularly when used over a prolonged period. In some examples, the filtered airflow may have an exit velocity of no more than 0.5 m/s.

As the jet of filtered airflow travels from the nozzle, surrounding unfiltered air is entrained in the airflow. The jet therefore comprises an outer region or entrained region that comprises a mix of filtered and unfiltered air, and an inner region or non-entrained region that comprises only filtered air. The applicant has observed that, whilst the jet diverges as it travels from the nozzle, the non-entrained region of the airflow converges. The air purifier may therefore be located within a set distance from the user so as to ensure that the non-entrained region has a minimum size at the face of the user, thereby improving the quality of the breathed air.

The applicant has further observed that the length of the non-entrained region (i.e. the distance between the nozzle and the convergent point of the non-entrained region) is proportional to the diameter of the outlet of the nozzle. Moreover, at these sort of flow velocities, the length of the non-entrained region was found to be approximately four times the diameter of the outlet. Accordingly, an air purifier having a larger diameter outlet may be located further from the user. Conversely, an air purifier having a smaller diameter outlet may be located closer to the user.

Interestingly, and somewhat counterintuitively, the length of the non-entrained region was found to be relatively insensitive to the exit velocity of the airflow. As the exit velocity of the airflow increases, one might think that the length of the non-entrained region would increase, i.e. the non-entrained region would extend further from the nozzle. However, it transpires that, as the exit velocity of the airflow increases, entrainment and mixing of the surrounding air is more aggressive and thus the non-entrained region converges more rapidly. As a result, the length of the non-entrained region is largely unchanged. Accordingly, irrespective of the distance of the air purifier from the user, the same relatively low exit velocity may be employed.

The exit velocity of the filtered airflow at the outlet may be no less than 0.1 m/s. Owing to the relatively low velocity of the filtered airflow, air currents within the room may deflect the path of the filtered airflow. By ensuring that the exit velocity is at least 0.1 m/s, deflection in the path of the airflow due to room currents may be mitigated.

The velocity of the airflow emitted from the air purifier may decrease as it travels from the air purifier to the user. This decrease in velocity may depend on the distance of the air purifier from the user. Accordingly, the control unit may control the blower such that the exit velocity depends on the distance of the air purifier from the face of the user. In particular, the control unit may control the blower such that the exit velocity of the airflow is increased in response to an increase in the distance of the air purifier from the face of the user. As a result, the efficacy of the air purifier may be improved without impacting comfort.

The control unit may be operable to identify a breathing zone of the detected face, which comprises the mouth and nose of the detected face. The control unit may then be operable to control the actuator assembly such that the filtered airflow is directed at the centre of the breathing zone. This then has the advantage that filtered air is purposively directed at that region of the face where it is most important, namely the mouth and nose. As a result, the efficacy of the air purifier may be improved. Additionally, by specifically targeting the breathing zone of the face, a smaller jet may be emitted by the air purifier and thus the delivery of filtered air may be achieved more efficiently.

The breathing zone may have a centre located between the nose and mouth of the detected face. Additionally or alternatively, the breathing zone may have a diameter of no greater than 150 mm. As noted in the preceding paragraph, by targeting the nozzle at a relatively small breathing zone that nevertheless encompasses the nose and mouth, a jet of non-entrained filtered air may be delivered at the breathing zone more efficiently, e.g. using a smaller nozzle and a lower flow rate.

The outlet may have a diameter or an equivalent diameter (if non-circular in shape) of between 20 mm and 150 mm. The outlet is therefore relatively small in comparison to that of a conventional air purifier. As a result, it is possible to achieve a relatively small and compact air purifier. The air purifier may therefore be located relatively close to a user, whilst remaining discreet and unobtrusive. Moreover, the air purifier may be more easily stored and transported. As noted above, the length of the non-entrained region is proportional to the diameter of the outlet. By employing an outlet of larger diameter (e.g. 150 mm), the length of the non-entrained region is longer. Accordingly, when the air purifier is placed at a set distance from the user, the size of the non-entrained region at the point of contact with the face will be larger and thus the efficacy of the air purifier may be improved. By employing an outlet of smaller diameter (e.g. 20 mm), a more compact air purifier may be achieved. As a result, the air purifier may be more easily ported, e.g. carried in a bag or the like. Moreover, should the air purifier include a cooling mode (discussed below in further detail), higher exit velocities can be achieved more easily with a nozzle having a smaller diameter. In particular, a given increase in exit velocity may be achieved by a smaller increase in the speed of the blower.

The actuator assembly may be controllable to adjust a yaw and a pitch of the nozzle to adjust the direction of the filtered airflow. For example, the actuator assembly may comprise a first transmission driven by a first motor to adjust the yaw of the nozzle, and a second transmission driven by a second motor to adjust the pitch of the nozzle. By adjusting both the pitch and yaw of the nozzle, the air purifier is able to accurately track the face of the user.

The actuator assembly may be controllable to adjust the yaw through an angle range of at least 90 degrees, and to adjust the pitch through an angle range of at least 60 degrees. Moreover, the actuator assembly may be controllable to adjust the yaw through an angle range of at least 120 degrees, and to adjust the pitch through an angle range of at least 90 degrees. As a result, the air purifier is able to track the face of the user over a relatively large range of motion.

The outlet of the nozzle may be non-annular in shape. As a result, a solid column of filtered air is emitted from the nozzle. This then has the advantage of reducing the entrainment of surrounding air and thus improving the purity of the air delivered to the face of the user.

The control unit may be operable in one of a purification mode and a cooling mode, and the control unit may control the blower such that the exit velocity of the filtered airflow has a maximum that is no greater than 1 m/s in purification mode, and is no less than 2 m/s in cooling mode. This then has the advantage that the air purifier may function as both an air purifier (purification mode) and a personal cooling fan (cooling mode). In purification mode, the air purifier emits a low velocity jet that is not intended to cool the user, but is instead intended to create a bubble of clean air at the face of the user. By contrast, in cooling mode, the air purifier emits a higher velocity airflow that is intended to cool the user. In purification mode, the air purifier tracks and targets the face of the user. In cooling mode, the air purifier may likewise track and target the face of the user. Alternatively, tracking of the face may be suspended in cooling mode and the direction of the airflow, together with perhaps the velocity of the airflow, may be controlled manually by a user.

The image sensor may be a thermal image sensor. This then has at least two advantages. First, the privacy of the user may be better protected. In particular, by using a thermal image sensor that senses only body heat, the control unit may detect a face in the image data but not the identity of the face. Second, the control unit may detect a face in image data captured in low-light conditions, such as at night when a user is sleeping.

The present invention also provides an air purifier comprising: a blower to generate an airflow; a filter to remove pollutants from the airflow; a nozzle comprising an outlet through which the filtered airflow is emitted; an actuator assembly to adjust the direction of the filtered airflow; an image sensor to capture image data; and a control unit, wherein the control unit is operable in one of a purification mode and a cooling mode, the control unit detects a face in the image data and controls the actuator assembly such that the filtered airflow is directed at the face when operating in purification mode, and the control unit controls the blower in both purification mode and cooling mode such that a maximum exit velocity of the filtered airflow in cooling mode is at least double a maximum exit velocity of the filtered airflow in purification mode.

The air purifier is therefore operable in one of two modes: purification and cooling.

In purification mode, the air purifier directs a jet of filtered air at the face of user. The air purifier automatically tracks the face of the user in purification mode. In particular, the control unit detects the face in the image data captured by the image sensor, and controls the actuator assembly such that the filtered airflow is directed at the detected face. Consequently, as the face of the user moves, the air purifier continues to direct a jet of filtered airflow at the face. In addition to tracking the face of the user, the control unit controls the blower such that the exit velocity of the filtered airflow is relatively low. As a result, the velocity of the filtered airflow at the face of the user is similarly low. This then has the benefit that the air purifier is less likely to cause discomfort, particularly when used over a prolonged period.

In purification mode, the airflow emitted by the air purifier is not intended to cool the user, but is instead intended to create a bubble of clean air at the face of the user. By contrast, in cooling mode, the air purifier emits a higher velocity airflow (at least double that in purification mode) that is intended to cool the user. In purification mode, the air purifier tracks and targets the face of the user. In cooling mode, the air purifier may likewise track and target the face of the user. Alternatively, tracking of the face may be suspended in cooling mode and the direction of the airflow, together with perhaps the velocity of the airflow, may be controlled manually by a user.

The maximum exit velocity of the filtered airflow in cooling mode may be at least three times greater than the maximum exit velocity of the filtered airflow in purification mode. As a result, a greater rate of cooling may be achieved in cooling mode.

### BRIEF DESCRIPTION OF THE DRAWINGS

FIG. 1 is a perspective view of an air purifier;

FIG. 2 is a sectional view through the air purifier;

FIG. 3 is a block diagram of electrical components of the air purifier;

FIG. 4 illustrates the breathing zone of a user; and

FIG. 5 is a side view of an airflow emitted by the air purifier.

### DETAILED DESCRIPTION OF THE INVENTION

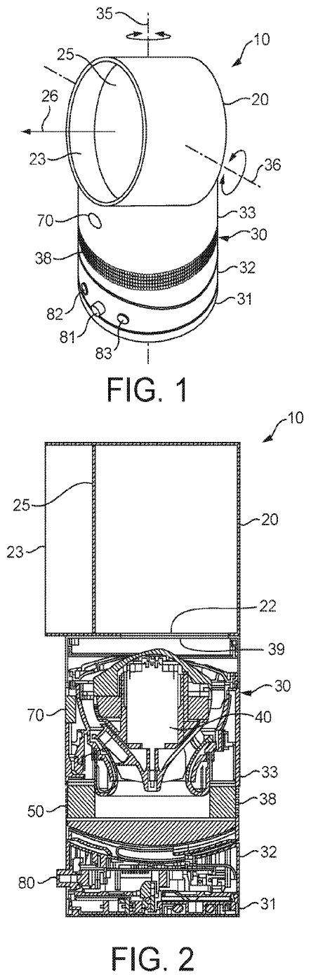

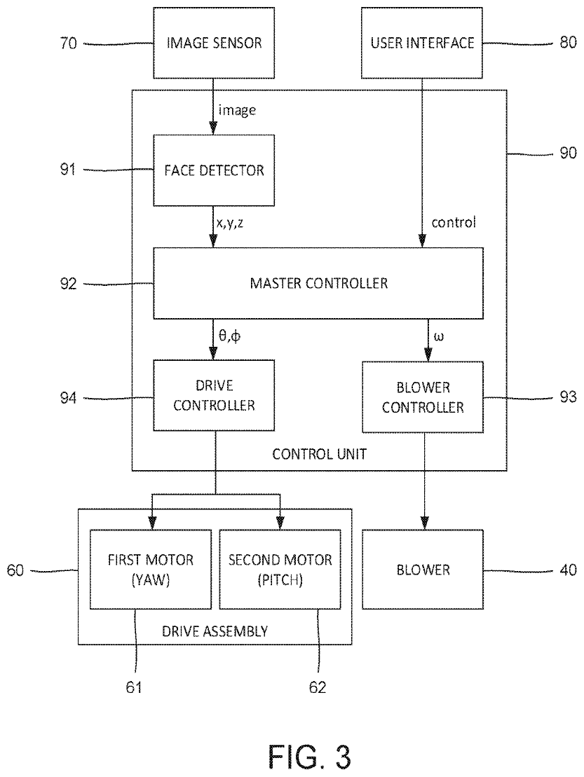

The air purifier 10 of FIGS. 1 to 3 comprises a nozzle 20 attached to a main body 30.

The nozzle 20 comprises a cylindrical wall that is open at a first end and is closed at a second opposite end. The open end defines an outlet 23 of the nozzle 20. An opening in a side of the wall defines an inlet 22 of the nozzle 20. The nozzle 20 further comprises a mesh screen or grille 25 located between the inlet 22 and the outlet 23. The screen 25 acts to prevent foreign objects from falling into the main body 30.

The main body 30 is generally cylindrical in shape and comprises a lower portion 31, a middle portion 32 and an upper portion 33. The lower portion 31 serves as a base for the main body 30 and thus a base for the air purifier 10. The middle portion 32 is attached to the lower portion 31 and is moveable relative to the lower portion 31 about a first rotational axis 35. The upper portion 33 is attached to the middle portion 32 and is moveable relative to the middle portion 32 about a second rotational axis 36. The upper portion 33 is therefore moveable relative to the lower portion 31 about both the first rotational axis 35 and the second rotational axis 36.

The first rotational axis 35 and the second rotational axis 36 are orthogonal. Moreover, one of the two rotational axes extends vertically, and the other of the two rotational axes extends horizontally. In this particular embodiment, the first rotational axis 35 extends vertically and the second rotational axis 36 extends horizontally.

The nozzle 20 is fixedly attached to the upper portion 33 of the main body 30 and may be regarded as having a central axis 26 along which an airflow is emitted from the outlet 23 of the nozzle 20. Rotation of the upper portion 33 of the main body 30 about the first rotational axis 35 therefore causes the central axis 26 of the nozzle 20 to precess about the first rotational axis 35. Likewise, rotation of the upper portion 33 about the second rotational axis 36 causes the central axis 26 of the nozzle 20 to precess about the second rotational axis 36. Rotation about the first rotational axis 35 may therefore be said to adjust the yaw of the nozzle 20, and rotation about the second rotational axis 36 may be said to adjust the pitch of the nozzle 20. In the present embodiment, the second rotational axis 36 extends through the nozzle 20 and may intersect the central axis 26 of the nozzle 20. As a result, the pitch of the nozzle 20 may be adjusted without significantly changing the position of the nozzle 20.

The main body 30 further comprises an inlet 38, an outlet 39, a blower 40, a filter 50, an actuator assembly 60, an image sensor 70, a user interface 80, and a control unit 90.

The inlet 38 and the outlet 39 are provided in the upper portion 33 of the main body 30. The inlet 38 comprises a plurality of apertures formed in a side wall of the upper portion 33, and the outlet 39 comprises an aperture formed in a top wall of the upper portion 33.

The blower 40 is housed within the upper portion 33 of the main body 30 and generates an airflow through the main body 30. More particularly, the airflow is drawn in through the inlet 38 in the main body 30, and is emitted from the outlet 39 of the main body 30 and into the nozzle 20.

The filter 50 is housed within the upper portion 33 and acts to filter the airflow moving through the main body 30. In the present embodiment, the filter 50 is located upstream of the blower 40. However, the filter 50 might equally be located downstream of the blower 40. Moreover, the main body 30 may comprise a filter located upstream of the blower 40 and a further filter located downstream of the blower 40.

The actuator assembly 60 is housed within the middle portion 32 of the main body 30 and is operable to move the middle portion 32 relative to the lower portion 31, and to move the upper portion 33 relative to the middle portion 32. The actuator mechanism 60 is therefore operable to adjust the direction of the nozzle 20. The actuator assembly 60 comprises a first motor 61, a second motor 62, a first transmission (not shown) and a second transmission (not shown). The first transmission transmits torque generated by the first motor 61 into rotational movement of the middle portion 32 relative to the lower portion 31 about the first rotation axis 35. The first motor 61 is therefore operable to adjust the yaw of the nozzle 20. The second transmission transmits torque generated by the second motor 62 into rotational movement of the upper portion 33 relative to the middle portion 32 about the second rotational axis 36. The second motor 62 is therefore operable to adjust the pitch of the nozzle 20. Each of the transmissions may comprise an arrangement of gears or the like for transmitting the torque into the required rotational movement.

The actuator assembly 60 may be operable to adjust the yaw of the nozzle through an angle range of at least 90 degrees, and to adjust the pitch through an angle range of at least 60 degrees. Moreover, the actuator assembly 60 may be operable to adjust the yaw through an angle range of at least 120 degrees, and to adjust the pitch through an angle range of at least 90 degrees. As explained below, the actuator assembly 60 is controlled by the control unit 90 such that the nozzle 20 tracks the face of a user. By ensuring that the yaw and pitch of the nozzle 20 can be adjusted through a relatively large range of angles, the nozzle 20 is able to track the face of the user over a relatively large range of motion.

The image sensor 70 is provided on the upper portion 33 of the main body 30 and captures image data of a scene located in front of the nozzle 20. Since the image sensor 70 is provided on the upper portion of the main body 30, the image sensor moves with the nozzle 20. The image sensor 70 is intended to capture image data of a user of the air purifier 10. Moreover, the image sensor 70 is intended to capture image data from which a face of the user can be detected. Accordingly, the image sensor 70 may be any sensor capable of capturing image data from which a face can be discerned. For example, the image sensor 70 may sense electromagnetic (e.g. visible, infrared, microwave, radio) or acoustic waveforms (e.g. time-of-flight). In some examples, the image sensor may be an optical sensor (e.g. RGB camera), a thermal sensor (e.g. IR camera), or a lidar sensor. The image sensor 70 may comprise a 2D array of pixel elements that capture 2D image data. Alternatively, the image sensor may comprise a 1D array of pixel elements, or indeed a single pixel element, that is scanned horizontally and/or vertically to capture 2D image data. In the present embodiment, the image sensor 70 is a thermal image sensor. This then has at least two benefits. First, the privacy of the user may be better protected. In particular, by using a thermal image sensor that senses only body heat, a face may be detected in the image data but not the identity of the face. Second, a face may be detected in image data captured in low-light conditions, such as at night when a user is sleeping.

The user interface 80 comprises one or more user-operable controls 81,82,83 for controlling the operation of the air purifier 10. In the present embodiment, the user interface 80 comprises a dial 81 for controlling the speed of the blower 40, and thus the exit velocity of the airflow emitted from the nozzle 20. The user interface 80 further comprises a first button 82 for powering on and off the air purifier 10, and a second button 83 for switching between a cooling mode and a purification mode, which is described below in more detail. The user interface 80 may comprise additional or alternative controls. For example, the user interface 80 may comprise controls for switching between tracking and non-tracking and/or for manually adjusting the direction of the nozzle 20 when operating in non-tracking mode. In addition (or as an alternative) to the user interface 80, the main body 30 may comprise a wireless interface for receiving control data from a remote device operated by a user. For example, the remote device may comprise a dedicated remote control or a mobile device, such as a phone or tablet. A user is then able to control remotely the operation of the air purifier 10.

The control unit 90 is housed within the middle portion 32 of the main body 30 and is responsible for controlling the operation of the air purifier 10. The control unit 90 comprises a face detector 91, a master controller 92, a blower controller 93, and a actuator controller 94.

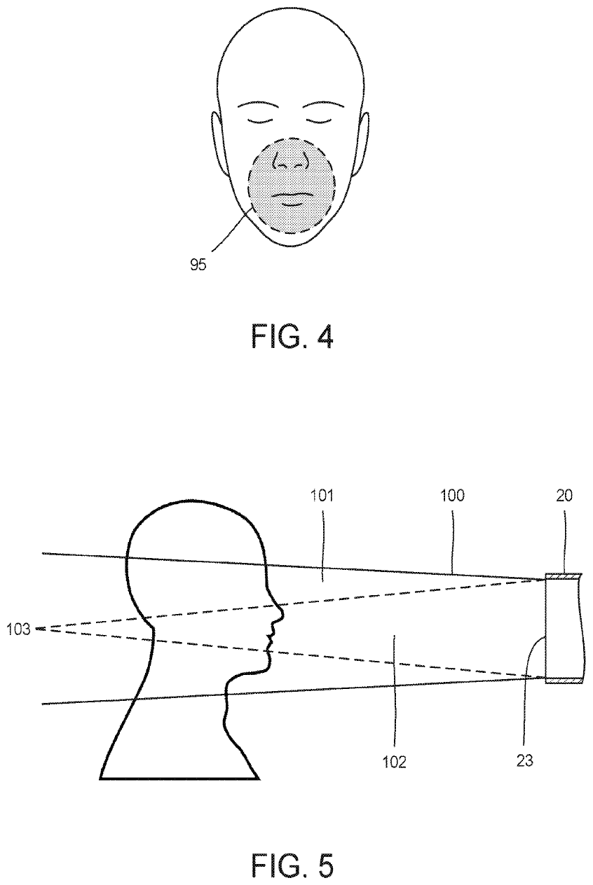

The face detector 91 receives image data from the image sensor 70 and detects a face within the image data. The face detector 91 employs object-class detection (e.g. using MediaPipe Face Mesh) to detect certain features (e.g. nose and mouth) within the detected face. The detected features are then used to determine the location of a breathing zone within the detected face. As shown in FIG. 4, the breathing zone 95 corresponds to an area of the detected face that comprises the nose and mouth. In this particular embodiment, the breathing zone 95 has a diameter (at the face of the user) of around 150 mm and is centred at a position between the nose and the mouth. In addition to detecting the location or centre of the breathing zone 95, the face detector 91 determines the distance of the breathing zone 95 from the air purifier 10. In this particular embodiment, the face detector 91 uses certain features within the detected face to determine the distance of the face, and thus the distance of the breathing zone 95, from the air purifier 10. For example, the face detector 91 may use the interpupillary distance of the detected face to determine the distance of the face from the air purifier 10. In alternative examples, the air purifier 10 may comprise an additional image sensor to provide stereoscopic image data from which the distance of the face, and thus the breathing zone, may be determined, or the air purifier 10 may comprise a distance sensor (e.g. time-of-flight ultrasonic sensor) for separately determining the distance of the face. The location of the breathing zone 95 is then output by the face detector 91 as a set of three-dimensional coordinates (x,y,z) to the master controller 92.

The master controller 92 receives control data from the user interface 80 and coordinate data from the face detector 91. In response to the received input data, the master controller 92 generates output data for controlling the blower 40 and the actuator assembly 60. In particular, the master controller 92 outputs a target speed value (ω) to the blower controller 93, and target yaw and pitch values (θ,ϕ) to the actuator controller 94. In response to the received speed value (ω), the blower controller 93 controls the speed of the blower 40. In response to the received yaw and pitch values (θ,ϕ), the actuator controller 94 controls the first and second motors 61,62 of the actuator assembly 60.

In the present embodiment, the control unit 90 comprises a face detector 91 together with a plurality of controllers 92,93,94. In alternative embodiments, the control unit 90 may comprise an alternative number and/or arrangement of components for performing the various functions of the control unit 90. Indeed, the control unit 90 could conceivably comprise a single controller. Accordingly, in a more general sense, the control unit 90 may be said to control the blower 40 and the actuator assembly 60 in response to image data received from the image sensor 70 and control data received from the user interface 80.

The air purifier 10 is operable in a purification mode and a cooling mode. A user is able to select the mode of operation via the user interface 80.

When operating in purification mode, the control unit 90 controls the blower 40 and the actuator assembly 60 such that the filtered airflow emitted from the nozzle 20 is directed at the breathing zone of the user. In particular, the control unit 90 detects the face of the user in the image data captured by the image sensor 70, and controls the actuator assembly 60 such that the nozzle 20 is directed at the breathing zone of the detected face. The control unit 90 tracks the face of the user such that, as the user moves, the filtered airflow emitted from the nozzle 20 continues to be directed at the breathing zone of the user. In addition to tracking the face of the user, the control unit 90 controls the blower 40 such that exit velocity of the filtered airflow at the outlet 23 of the nozzle 20 is relatively low. As a result, the velocity of the filtered airflow at the face of the user is similarly low. This then has the benefit that the air purifier 10 is less likely to cause discomfort to the user, particularly when used over a prolonged period. By contrast, if the velocity of the airflow were relatively high, the user may find that the continuous movement of air over the face is unpleasant. Additionally, the high movement of air may cause drying of the eyes and/or mouth.

Referring now to FIG. 5, as the filtered airflow 100 travels from the nozzle 20, surrounding unfiltered air is entrained in the airflow. The airflow 100 therefore comprises an outer region or entrained region 101 that comprises a mix of filtered and unfiltered air, and an inner region or non-entrained region 102 that comprises only filtered air. The applicant has observed that, whilst the airflow 100 diverges as it travels from the nozzle 20, the non-entrained region 102 of the airflow 100 converges.

The applicant has further observed that the length of the non-entrained region 102 (i.e. the distance between the nozzle 20 and the convergent point 103 of the non-entrained region 102) is proportional to the diameter of the outlet 23 of the nozzle 20. Moreover, at these sort of flow velocities, the length of the non-entrained region 102 was found to be approximately four times the diameter of the outlet 23. Accordingly, where the air purifier 10 is intended to be located further from the user, a nozzle 20 having a larger diameter outlet 23 may be employed. Conversely, where the air purifier 10 is intended to be located closer to the user, a nozzle 20 having a smaller diameter outlet 23 may be employed. Whilst the air purifier 10 of the present embodiment comprises a single nozzle 20, the air purifier 10 could conceivably comprise a plurality of interchangeable nozzles having outlets of different diameters.

Interestingly, and somewhat counterintuitively, the length of the non-entrained region 102 was found to be relatively insensitive to the exit velocity of the airflow 100. As the exit velocity of the airflow 100 increases, one might think that the length of the non-entrained region 102 would increase, i.e. that the non-entrained region 102 would extend further from the nozzle 20. However, it transpires that, as the exit velocity of the airflow 100 increases, entrainment and mixing of the surrounding air is more aggressive and thus the non-entrained region 102 converges more rapidly. As a result, the length of the non-entrained region 102 is largely unchanged. Accordingly, irrespective of the distance of the air purifier 10 from the user, the same relatively low exit velocity may be employed.

The applicant has observed that, in many instances, an airflow having a velocity greater than about 0.5 m/s at the face of the user can lead to discomfort, particularly over a prolonged period. However, the applicant has also observed that this velocity threshold is sensitive to the ambient conditions, particularly the ambient temperature and humidity. For example, in relatively warm and humid conditions, an airflow having a velocity of around 1.0 m/s at the face of the user was found to be reasonably comfortable. Accordingly, the control unit 90 may control the blower 40 such that the exit velocity of the filtered airflow at the outlet 23 of the nozzle 20 is no greater than 1 m/s. In some examples, and for reasons already noted, the filtered airflow may have an exit velocity of no more than 0.5 m/s.

The velocity of the airflow emitted from the air purifier 10 decreases as it travels from the air purifier 10 to the user. This decrease in velocity will depend on the distance of the air purifier 10 from the user. Accordingly, the control unit 90 may control the blower 40 such that the exit velocity depends on the distance of the air purifier 10 from the face of the user. In particular, the control unit 90 may increase the exit velocity of the airflow in response to an increase in the distance of the air purifier 10 from the face of the user. As a result, the efficacy of the air purifier 10 may be improved without impacting comfort.

Owing to the relatively low exit velocity of the filtered airflow, air currents within the room may deflect the path of the filtered airflow. Accordingly, the control unit 90 may control the blower 40 such that the exit velocity of the filtered airflow is no less than 0.1 m/s.

The diameter of the outlet 23 of the nozzle 20 may be between 20 mm and 150 mm. The outlet 23 is therefore relatively small in comparison to that of a conventional air purifier. As a result, it is possible to achieve a relatively small and compact air purifier. The air purifier 10 may therefore be located relatively close to a user, whilst remaining discreet and unobtrusive. Moreover, the air purifier 10 may be more easily stored and transported. As noted above, the length of the non-entrained region 102 is proportional to the diameter of the outlet 23. By employing an outlet 23 of larger diameter (e.g. 150 mm), a longer non-entrained region 102 is obtained. Accordingly, when the air purifier 10 is placed at a set distance from the user, the size of the non-entrained region at the point of contact with the face will be larger and thus the efficacy of the air purifier may be improved. By employing an outlet 23 of smaller diameter (e.g. 20 mm), a more compact air purifier may be achieved. As a result, the air purifier 10 may be more easily ported, e.g. carried in a bag or the like. Moreover, should the air purifier 10 include a cooling mode (discussed below in further detail), higher exit velocities can be achieved more easily with a nozzle having a smaller diameter. In particular, a given increase in exit velocity may be achieved by a smaller increase in the speed of the blower 40.

When operating in cooling mode, the control unit 90 controls the blower 40 such that the filtered airflow has a relatively high exit velocity at the outlet 23 of the nozzle 20. In purification mode, the airflow emitted by the air purifier 10 is not intended to cool the user. Instead, the filtered airflow is intended to create a bubble of clean air at the face of the user. By contrast, in cooling mode, the airflow emitted by the air purifier 10 is intended to cool the user. The exit velocity of the filtered airflow in cooling mode may therefore be at least double that in purification mode and may, in some examples, be at least three times that in purification mode.

Moreover, the exit velocity of the filtered airflow in cooling mode may be at least 2 m/s. As a result, effective cooling may be achieved.

The user interface 80 comprises a dial 81 which the user many operate in order to control the speed of the blower 40 and thus the exit velocity of the filtered airflow. Accordingly, when operating in purification mode and/or cooling mode, the user may vary the exit velocity of the filtered airflow. Nevertheless, the maximum exit velocity of the filtered airflow in purification mode may be no more than 1 m/s and, in some examples, no more than 0.5 m/s. The maximum exit velocity of the filtered airflow in cooling mode may then be double that in purification mode, and may be at least 2 m/s.

When operating in purification mode, the control unit 90 tracks and directs the nozzle 20 at the breathing zone of the user. In cooling mode, the control unit 90 may likewise track the breathing zone of the user. Alternatively, tracking may be suspended and the direction of the nozzle 20 may be controlled manually by the user, if required.

Conventional air purifiers typically provide bulk filtration of air within a room. Whilst this may lead to a reduction in the overall level of pollutants within the room, the level of pollutants may continue to be high at the location of a user. By contrast, the air purifier of the present invention directs a jet of filtered air at the face of user. As a result, the air breathed by the user may have a lower level of pollutants. Conceivably, a conventional air purifier may be directed at the face of a user. However, there are at least two difficulties with this. First, continual adjustment of the direction of the air purifier would be required to ensure that, as the user moves, the filtered air continues to be directed at the face of the user. However, continually adjusting the air purifier, at least manually, would not be a sustainable or realistic solution. Second, the airflow emitted from a conventional air purifier has a relatively high exit velocity. This relatively high exit velocity is required to ensure that the filtered air is circulated well within the room. If the exit velocity were relatively low, the filtered air would be localised around the air purifier, and thus the air purifier would provide relatively poor efficacy. However, as a consequence of the high exit velocity, directing the airflow of a conventional air purifier at the face of a user is likely to be uncomfortable, particularly over a prolonged period of time.

The air purifier 10 of the present invention automatically tracks the face of the user. In particular, the control unit 90 detects the face in the image data captured by the image sensor 70, and controls the actuator assembly 60 such that the nozzle 20 is directed at the detected face. Consequently, as the face of the user moves, the air purifier 10 continues to direct a jet of filtered air at the face without the need for manual intervention. In addition to tracking the face of the user, the filtered airflow emitted from the nozzle 20 has a relatively low exit velocity, e.g. no greater than 1 m/s. As a result, the velocity of the filtered airflow at the face of the user is similarly low. The air purifier 10 is therefore more comfortable, particularly when used over a prolonged period.

In the embodiment described above, the air purifier 10 is operable in a purification mode and a cooling mode. This then has the advantage that the air purifier 10 may function as both an air purifier (purification mode) and a personal cooling fan (cooling mode). However, conceivably, the air purifier 10 may be operable in a purification mode only. Moreover, in other alternatives, the air purifier 10 may comprise further operating modes. For example, the air purifier 10 may comprise a heating element and the air purifier 10 may be operable in a heating mode in which filtered airflow is heated.

The control unit 90 detects a face within the image data received from the image sensor 70. More particularly, the control unit 90 identifies a breathing zone within the detected face. The control unit 90 then controls the actuator assembly 70 such that the filtered airflow is directed at the centre of the breathing zone. This then has the benefit that the filtered airflow is purposively directed at that region of the user where it is most important, namely the mouth and nose. As a result, the efficacy of the air purifier 10 may be improved. Additionally, by specifically targeting the breathing zone of the user, a smaller jet may be emitted by the air purifier 10 and thus the delivery of filtered air may be achieved more efficiently. In spite of these benefits, the control unit 90 may detect a face within the image data without necessarily identifying the breathing zone. This in itself may have some advantages. For example, a simpler, cheaper image sensor may be used that is incapable of or is relatively poor at capturing the features of the face necessary to identify the breathing zone. Additionally or alternatively, a simpler object-classification algorithm may be used to detect the face and thus a less computationally powerful control unit may be employed.

The outlet 23 of the nozzle 20 is circular in shape. As a result, a solid column of filtered air is emitted from the nozzle 20. This then has the advantage of reducing the entrainment of surrounding air and thus improving the purity of the air that is delivered to the face of the user. Conceivably, the outlet 23 of the nozzle 20 may have an alternative shape which nevertheless continues to emit a solid column of air, e.g. oval or polygonal. The column of air will, however, eventually breakdown or collapse into a circular column of air. The use of a circular outlet is therefore likely to be more efficient. Nevertheless, alternative shapes may be employed, perhaps for aesthetic reasons. The outlet of a conventional air purifier may take the form of an annulus. An annular outlet encourages entrainment, which in turn provides better mixing and circulation of the filtered air with the surrounding air. With the air purifier of the present invention, on the other hand, entrainment with the surrounding air is undesirable. Accordingly, the outlet of the nozzle may be non-annular.

Whilst particular examples and embodiments have thus far been described, it should be understood that these are illustrative only and that various modifications may be made without departing from the scope of the invention as defined by the claims.

1. An air purifier comprising:
a blower to generate an airflow;
a filter to remove pollutants from the airflow;
a nozzle comprising an outlet through which the filtered airflow is emitted;
an actuator assembly to adjust the direction of the filtered airflow;
an image sensor to capture image data; and
a control unit to detect a face in the image data and to control the actuator assembly such that the filtered airflow is directed at the face,
wherein the filtered airflow has an exit velocity at the outlet of no greater than 1 m/s.

2. The air purifier as claimed in claim 1, wherein the exit velocity is no greater than 0.5 m/s.

3. The air purifier as claimed in claim 1, wherein the exit velocity is no less than 0.1 m/s.

4. The air purifier as claimed in claim 1, wherein the control unit determines a distance of the face, and the control unit controls the blower such that the exit velocity of the filtered airflow depends on the distance of the face.

5. The air purifier as claimed in claim 1, wherein the control unit is operable to identify a breathing zone of the detected face and to control the actuator assembly such that the filtered airflow is directed at a centre of the breathing zone, wherein the breathing zone comprises the nose and mouth of the detected face.

6. The air purifier as claimed in claim 5, wherein the breathing zone has a centre located between the nose and mouth of the detected face and/or a diameter of no greater than 150 mm.

7. The air purifier as claimed in claim 1, wherein the outlet has a diameter or equivalent diameter of between 20 mm and 150 mm.

8. The air purifier as claimed in claim 1, wherein the actuator assembly is controllable to adjust a yaw and a pitch of the nozzle.

9. The air purifier as claimed in claim 8, wherein the actuator assembly is controllable to adjust the yaw through an angle range of at least 90 degrees, and to adjust the pitch through an angle range of at least 60 degrees.

10. The air purifier as claimed in claim 1, wherein the outlet is non-annular in shape.

11. The air purifier as claimed in claim 1, wherein the control unit is operable in one of a purification mode and a cooling mode, and the control unit controls the blower such that the exit velocity of the filtered airflow has a maximum that is no greater than 1 m/s in purification mode, and is no less than 2 m/s in cooling mode.

12. The air purifier as claimed in claim 1, wherein the image sensor is a thermal image sensor.

13. An air purifier comprising:
a blower to generate an airflow;
a filter to remove pollutants from the airflow;
a nozzle comprising an outlet through which the filtered airflow is emitted;
an actuator assembly to adjust the direction of the filtered airflow;
an image sensor to capture image data; and
a control unit,
wherein the control unit is operable in one of a purification mode and a cooling mode, the control unit detects a face in the image data and controls the actuator assembly such that the filtered airflow is directed at the face when operating in purification mode, and the control unit controls the blower in both purification mode and cooling mode such that a maximum exit velocity of the filtered airflow in cooling mode is at least double a maximum exit velocity of the filtered airflow in purification mode.

14. The air purifier as claimed in claim 13, wherein the maximum exit velocity of the filtered airflow is no greater than 1 m/s in purification mode, or is no less than 2 m/s in cooling mode.

15. The air purifier as claimed in claim 13, wherein the filtered airflow has a minimum exit velocity of no less than 0.1 m/s in purification mode.

16. The air purifier as claimed in claim 13, wherein the control unit determines a distance of the face, and the control unit controls the blower in at least one of the purification mode and the cooling mode such that the exit velocity of the filtered airflow depends on the distance of the face.

17. The air purifier as claimed in claim 13, wherein, when operating in purification mode, the control unit is operable to identify a breathing zone of the detected face and to control the actuator assembly such that the filtered airflow is directed at a centre of the breathing zone, wherein the breathing zone comprises the mouth and nose of the detected face.

18. The air purifier as claimed in claim 17, wherein the breathing zone has a diameter of no greater than 150 mm.

19. The air purifier as claimed in claim 13, wherein the outlet has a diameter of between 20 mm and 150 mm.

20. The air purifier as claimed in claim 13, wherein the actuator assembly is controllable to adjust a pitch and a yaw of the nozzle.

21. The air purifier as claimed in claim 20, wherein the actuator assembly is controllable to adjust the pitch through an angle range of at least 90 degrees, and to adjust the pitch through an angle range of at least 60 degrees.

22. The air purifier as claimed in claim 13, wherein the outlet is non-annular in shape.

23. The air purifier as claimed in claim 13, wherein the image sensor is a thermal image sensor.
